(19) United States
(12) Patent Application Publication
(10) Pub. No.: US 2024/0164382 A1
(43) Pub. Date: May 23, 2024

(54) Method of promoting rice growth using Artificial Humic Acid Synthesized by Catalysis of Nanoscale Ferric Oxide

(71) Applicant: Jiangnan University, Wuxi (CN)

(72) Inventors: Zhenyu WANG, Wuxi (CN); Xiaona LI, Wuxi (CN); Fei LIAN, Wuxi (CN); Chuanxi WANG, Wuxi (CN); Le YUE, Wuxi (CN)

(21) Appl. No.: 18/426,497

(22) Filed: Jan. 30, 2024

Related U.S. Application Data
(63) Continuation of application No. PCT/CN2022/128275, filed on Oct. 28, 2022.

(30) Foreign Application Priority Data
Jan. 4, 2022 (CN) 2022100017577

(51) Int. Cl. A01N 43/84; A01C 1/00; B01J 23/745; B01J 35/45; C07D 413/14

(52) U.S. Cl. CPC A01N 43/84; A01C 1/00; B01J 23/745; B01J 35/45; C07D 413/14

(57) ABSTRACT

The present disclosure discloses a method of promoting rice growth using artificial humic acid synthesized by catalysis of nanoscale Ferric oxide, belonging to the field of nanoscale agriculture. The present disclosure discloses a method for synthesizing artificial humic acid from waste biomass with catalysis of transition metal, including the following steps: adding a mixed solution of an alkali, a transition metal catalyst and water into a biomass raw material, and carrying out catalytic reaction at 160-250° C. for 6-48 hours. The artificial humic acid can accelerate germination of rice, effectively promote growth of the rice (the root activity is improved by 166.76% and the net photosynthetic rate is improved by 72.08%), improve the absorption of water and nutrients and the transport of nutrients by rice roots and improve the ability of rice to resist oxidative stress and salt stress.

### TECHNICAL FIELD

The present disclosure relates to a method of promoting rice growth using artificial humic acid synthesized by catalysis of nanoscale Ferric oxide, belonging to the field of nanoscale agriculture.

### BACKGROUND

Carbon is an essential macroelement for plant growth. It is estimated that the carbon content in the terrestrial carbon pool is about 10¹⁶ tons, which is 1.15×10⁴ times that of the atmospheric carbon pool, and the terrestrial carbon pool plays an important role in global carbon supply and balance. The active carbon pool in the terrestrial carbon pool is mainly organic carbon, which is about 1.50×10¹² tons and plays an important role in maintaining soil structure, improving soil fertility and supporting crop growth. In the organic carbon pool, humic acid is the most active and abundant part. The humic acid promotes the crop growth by providing nutrients and improving photosynthesis and disease resistance, can improve water holding capacity and microbial activity of soil, improve nitrogen mineralization and soil texture, and improve nutrient effectiveness, thereby contributing to growth of plant roots. In addition, the humic acid can also slow down the decomposition of fertilizers and improve the nutrient utilization efficiency of crops.

The natural humus layer in the soil is generally formed slowly from leaf-litter or plant residues through microbial metabolism. However, the formation of natural humic acid takes hundreds or thousands of years. Moreover, the humification of biomass requires an anaerobic or extreme temperature environment, so the distribution of humus-rich soil in China is extremely uneven, and most of the soil has insufficient content of the humic acid. Usually, it takes a lot of material and financial resources to extract the natural humic acid from nature. All the above factors greatly limit the action of the humic acid in agricultural production. It is known that more than 220 billion tons of carbon enters plants through photosynthesis every year, but most of this carbon will be wasted with waste biomass, and only a small part can return to the soil. In addition, improper disposal of the waste biomass brings economic losses and environmental burdens. Therefore, how to reuse this waste biomass containing a lot of effective carbon is a research hotspot.

Hydrothermal humification of carbon in biomass by chemical methods to generate artificial humic acid is one of the effective methods. Studies have shown that the artificial humic acid synthesized by the hydrothermal method has similar surface morphology and structural properties to the natural humic acid, and has certain application potential in promoting plant nutrient absorption. However, the synthesis of the humic acid by the conventional hydrothermal method requires extreme environments with strong acids or strong bases, and has the disadvantages of low yield and violent reaction. In addition, the composition of the artificial humic acid has not been reported yet, and its key substances for promoting plant growth are still undefined.

### SUMMARY

### Technical Problems

The synthesis of artificial humic acid by the conventional hydrothermal method has the problems of too violent reaction, low product recovery, less active ingredients for promoting plant growth, and undefined composition of the artificial humic acid.

### Technical Solutions

In order to solve the above problems, in the present disclosure, the artificial humic acid is synthesized by using waste biomass as a raw material and transition metal as a catalyst; the composition and synthesis process of the artificial humic acid are identified by UPLC-MS/MS; and then, the artificial humic acid is used for the plant growth, and its growth promoting effect is tested. The method for synthesizing the artificial humic acid according to the present disclosure is green and simple, is easy to apply and operate in agriculture and has good effect.

A first object of the present disclosure is to provide a method for synthesizing artificial humic acid from waste biomass with catalysis of transition metal, including the following steps:
adding a mixed solution of an alkali, a transition metal catalyst and water into a biomass raw material, and carrying out catalytic reaction at 160-250° C. for 6-48 hours; and after the completion of the reaction, carrying out solid-liquid separation on a reaction product, and filtering an obtained liquid to obtain the artificial humic acid.

In an embodiment of the present disclosure, the biomass includes bulk farmland waste biomass and greening waste biomass, where the bulk farmland waste biomass includes straws, rice husks, peanut shells, etc.; and the greening waste biomass includes branches, leaves, etc.

In an embodiment of the present disclosure, the alkali includes potassium hydroxide and sodium hydroxide.

In an embodiment of the present disclosure, the transition metal catalyst is nanoscale ferric oxide. The transition metal catalyst has a nanoscale particle size (<100 nm).

In an embodiment of the present disclosure, a ratio of the biomass raw material to the transition metal catalyst is 2.5 to 3.5:1, further preferably 3:1.

In an embodiment of the present disclosure, a ratio of the biomass raw material to the water is 1 g:15 to 25 mL, further preferably 1 g:20 mL.

In an embodiment of the present disclosure, a concentration of the alkali in the water is not higher than 0.01 g/mL.

In an embodiment of the present disclosure, the filtering is filtering with a filter membrane less than 0.45 μm.

A second object of the present disclosure is to provide artificial humic acid prepared by the method of the present disclosure.

In an embodiment of the present disclosure, the artificial humic acid contains phytohormone substances such as coumaric acid and isocitric acid.

A third object of the present disclosure is to provide a method for promoting plant growth by using the artificial humic acid of the present disclosure, including: adding the artificial humic acid in a plant seed germination or plant growth process and carrying out culture.

In an embodiment of the present disclosure, the adding the artificial humic acid in a plant seed germination process specifically includes: culturing the plant seeds in a solution with the artificial humic acid at room temperature until the plant seeds germinate.

In an embodiment of the present disclosure, the plant seeds need sterilization before germination; and where the sterilization uses a 5% (v:v) H₂O₂ solution.

In an embodiment of the present disclosure, the adding the artificial humic acid in a plant growth process specifically includes: culturing rice seeds with uniform germination conditions in an environment containing soil and the artificial humic acid; and where a ratio of the artificial humic acid to the soil is 1/30 (mL/g).

In an embodiment of the present disclosure, the plant seeds include rice seeds; the plant includes rice plants.

### Beneficial Effects

(1) The raw materials for preparing the artificial humic acid in the present disclosure come from a wide range of sources, mainly including biomass waste, and the preparation method is simple and easy to implement and has the characteristics of low carbon and environmental friendliness.

(2) The artificial humic acid prepared by the present disclosure has similar morphological structure to natural humic acid.

(3) According to the present disclosure, nanoscale transition metal is used as the catalyst to accelerate the hydrothermal humification process of biomass macromolecules, which improves the degradation rate of the biomass (to above 14%) and promotes the synthesis of growth hormone analogs (e.g., isocitric acid, coumaric acid, etc.) in the artificial humic acid.

(4) The artificial humic acid synthesized with catalysis of the nanoscale transition metal can accelerate germination of rice (the germination rate is improved by 15%), effectively promote growth of the rice (the root activity is improved by 166.76% and the net photosynthetic rate is improved by 72.08%), improve the absorption of water and nutrients and the transport of nutrients by rice roots and improves the ability of rice to resist oxidative stress and salt stress. The method is simple in preparation, convenient to operate and easy for popularization and application.

### BRIEF DESCRIPTION OF FIGURES

FIG. 1 is a scanning electron microscope (SEM) image of natural humic acid extracted from black soil, artificial humic acid obtained by treatment with KOH in Comparative Example 2, artificial humic acid obtained by treatment with KOH+FeCl₃ in Comparative Example 1 and artificial humic acid obtained by treatment with KOH+Fe₂O₃ in Example 1;

FIG. 2 is a picture showing functional groups on the surface of artificial humic acid;

FIG. 3 is a schematic diagram showing a synthesis process of artificial humic acid;

FIG. 4 shows common substances in artificial humic acids obtained in Example 1, Comparative Example 1 and Comparative Example 2;

FIG. 5 shows particular substances in artificial humic acid obtained in Example 1;

FIG. 6 shows particular substances in artificial humic acid obtained in Comparative Example 1;

FIG. 7 shows particular substances in artificial humic acid obtained in Comparative Example 2;

FIG. 8 shows real pictures of effects of adding artificial humic acid on rice germination; and

FIG. 9 shows real pictures of effects of adding artificial humic acid on plant growth.

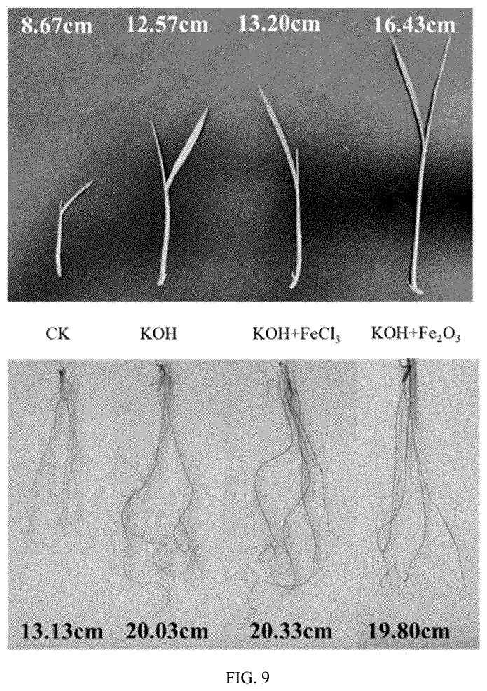

### DETAILED DESCRIPTION

Preferred examples of the present disclosure will be described below. It should be understood that the examples are intended to better explain the present disclosure and are not intended to limit the present disclosure.

Rice seeds used in the examples and comparative examples are purchased from Yueyou 9113 rice hybrid seeds from Wuxi City, Jiangsu Province.

Test Method:

Determination of TOC content in artificial humic acid: A liquid product of hydrothermal reaction is filtered by a 0.25 um hydrophilic filter membrane, the filtrate is diluted 300 times with deionized water, and then the TOC content is determined by a TOC analyzer (varioTOC cube/selet, elementar, Germany).

Test of germination rate: Rice seeds with uniform conditions are sterilized with a 5% (v:v) H₂O₂ solution for 10 minutes and placed in a seedling tray filled with a vermiculite culture medium, and 0.5 mL of artificial humic acid is added. Each treatment is repeated 3 times. 10 rice seeds are placed in each square. Within 7 days of culture, the number of rice seeds germinated in each square is recorded every day, and the germination rate is calculated.

Test of plant height, dry weight, fresh weight, nutrient contents, photosynthetic rate, soluble sugar content and soluble protein content: Rice seedlings with uniform germination conditions are cultured, and the photosynthetic rate is determined by a photosynthetic apparatus. After 30 days of culture, rice plants are harvested. After the rice plants are washed with clean water, the length of aerial parts (plant height) and the root length are determined with a ruler, and fresh weights of the aerial parts and the roots of the rice are measured and recorded. These samples are placed into paper envelopes, de-enzymed in an oven at 105° C. for half an hour and then dried at 60° C. to constant weight. The dry weights of the samples are recorded.

Determination of nutrient content by ICP-MS: The dry samples of the aerial parts and the roots are cut into small pieces. 25 mg of the dry sample is placed in a digestion tube, 3 mL of HNO₃ and 3 mL of H₂O are added, and the sample is digested in a microwave digestion system (MARS 6, CEM, USA). The solution is cooled, then transferred into a 50 mL centrifuge tube, and adjusted to a volume of 50 mL. Then, the contents of elements P and K in the aerial parts and the roots of the plants are determined with an inductively coupled plasma mass spectrometer (iCAP-TQ, Thermo Fisher, Germany).

Determination of content of N with Kjeldahl apparatus: 0.3 to 0.5 g of dry sample is placed in a large test tube. 0.2 g of copper sulfate and 3 g of potassium sulfate (Chinese Standard) (copper sulfate:potassium sulfate=1:15) are added. 10 ml of concentrated sulfuric acid is added. The large test tube is placed on a digestion furnace in a fume hood and heated such that the sample is digested. The digested sample is distilled in the Kjeldahl apparatus, and titrated with 0.05 M standard hydrochloric acid. The content of N is calculated according to specific formula (1) below:

N(%)=(1.401×M/W)×(V−V₀) (1)

In the formula (1), M=molar concentration of standard acid; W=sample weight (g); V₀=consumption of standard acid for titration of blank sample (mL); and V=consumption of standard acid for titration of sample (mL).

Determination of soluble sugar content by anthrone method: 20 mg (W) of dry sample is placed in a 1.5 mL centrifuge tube. 80% ethanol is added, and the sample is treated in a water bath for 30 minutes. The mixture is centrifuged, and the supernatant is taken. A small amount of activated carbon is added, and the resulting mixture is treated in a water bath until is decolorized. 5 mL of anthrone reagent is added to 1 mL of the decolorized supernatant, and the absorbance at the wavelength of 625 nm is measured. The soluble sugar content is calculated according to formula (2) below:

soluble sugar content (%)=[(C*V/a)/W]*10⁻⁴ (2)

In the formula (2), C=concentration of soluble sugar in extracting solution, available from standard curve; W=sample weight (g); V=total volume of extracting solution (ml); and a=volume used in determination.

Determination of soluble protein: 0.5 g of dry sample is measured and placed in a mortar, and 5 ml of pH=7.8 phosphate buffer is added. The mixture is ground in an ice bath, and the homogenate is poured into a centrifuge tube and treated in a refrigerated centrifuge for 20 min (10000×g). 20 μL (V) of supernatant (enzyme solution)+3 mL of G-250 is allowed to stand for 2 min and subjected to colorimetric assay at 595 nm. (20 μL of buffer+3 mL of G-250) is prepared as a blank. The soluble protein content is calculated according to formula (3) below:

soluble protein (mg/Gfw)=(C×V/Va)/W (3)

In the formula (3), C=concentration of soluble protein in extracting solution, available from standard curve; W=sample weight (g); V=total volume of extracting solution (ml); and Va=volume used in determination.

### Example 1

A method for synthesizing artificial humic acid by using nanoscale ferric oxide (treatment with KOH+Fe₂O₃) included the following steps:

3 g of corn straw was measured and added to a 100 ml reactor as a reaction precursor substance, followed by the addition of a mixed solution of 0.62 g of potassium hydroxide (analytically pure), 1 g of nanoscale ferric oxide (5 nm) and 60 mL of deionized water. The mixture was placed in an oven and subjected to catalytic reaction at 200° C. for 24 h. After the completion of the reaction, the resulting mixture was cooled to room temperature, and the reactor was opened. The liquid was separated from solid residues. The liquid was filtered with a 0.22 um filter membrane to obtain artificial humic acid.

### Comparative Example 1

Treatment with KOH+FeCl₃:

The nanoscale ferric oxide in Example 1 was replaced with ferric chloride, and the other conditions were the same as in Example 1 to obtain artificial humic acid.

### Comparative Example 2

Treatment with KOH:

The nanoscale ferric oxide in Example 1 was omitted, and the other conditions were the same as in Example 1 to obtain artificial humic acid.

The artificial humic acids obtained in Example 1 and Comparative Examples 1 and 2 were subjected to performance test. The test results are as follows:

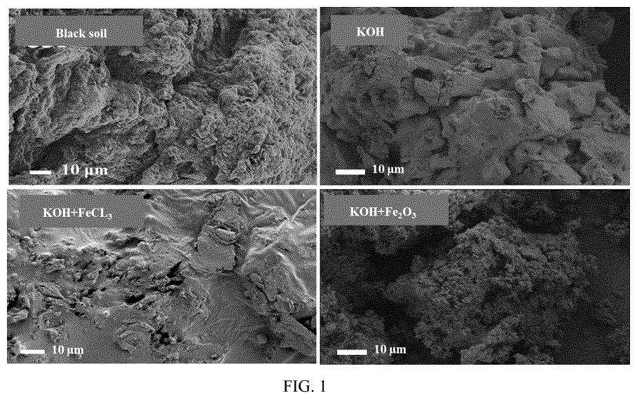

As can be seen from FIG. 1, the artificial humic acids obtained in Example 1 and Comparative Examples 1 and 2 had similar structure to that of natural humic acid extracted from black soil.

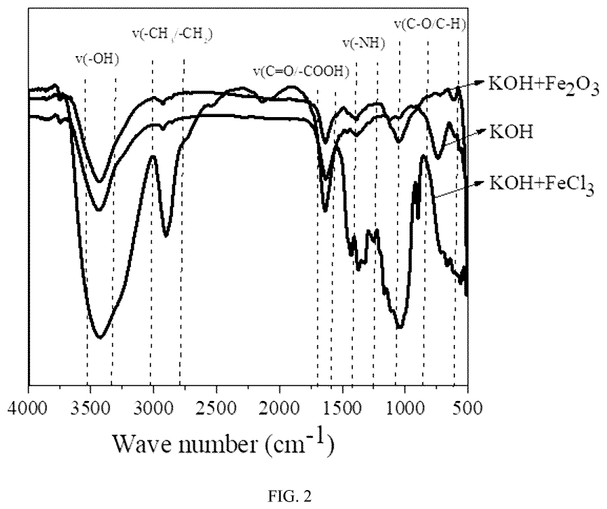

As can be seen from FIG. 2, compared with Comparative Example 2 in which no catalyst is added, the artificial humic acid prepared by using the ferric chloride as the catalyst has higher contents of —CH₂ and —CH₃ groups, which indicates that the artificial humic acid in Comparative Example 1 contains more lipid substances. The artificial humic acid prepared by using the nanoscale ferric oxide as the catalyst in Example 1 has a smaller number of oxygen-containing functional groups, which indicates that Example 1 has a higher degree of the artificial humification and a higher content of C.

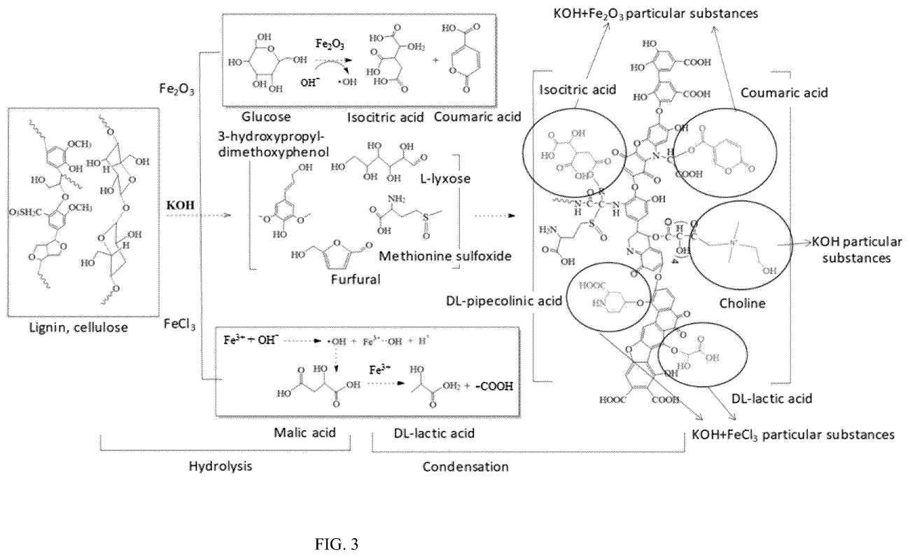

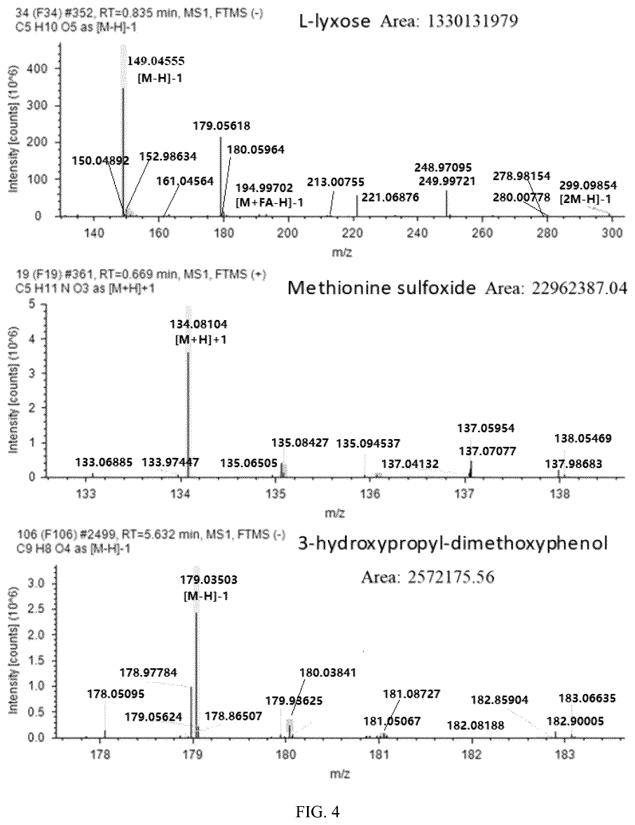

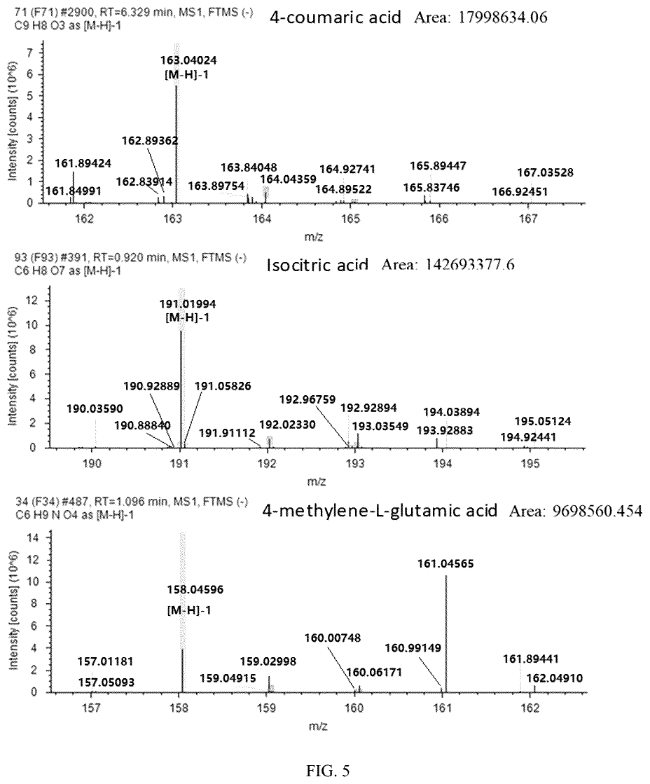

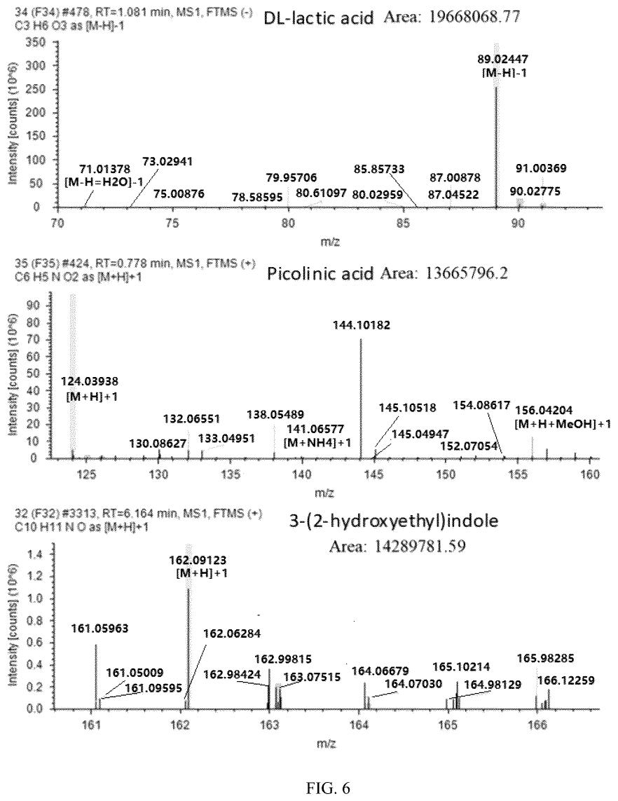

FIG. 3 is a schematic diagram showing a synthesis process of artificial humic acid. As can be seen from FIG. 3, the artificial humic acid contains methionine sulfoxide, which can promote germination of plant seeds (FIG. 4). Moreover, compared with Comparative Example 2 in which no catalyst is added, the KOH+Fe₂O₃ treatment group contains coumaric acid and isocitric acid (FIG. 5), which can promote plant growth. The KOH+FeCl₃ treatment group contains lactic acid and DL-pipecolinic acid (FIG. 6). However, it is difficult to extract these substances from natural humic acid.

### Example 2 Germination

A method for promoting rice seed germination by using artificial humic acid included the following steps:

Rice seeds were sterilized with 5% H₂O₂ for 15 min. The rice seeds were placed in 9 cm culture dishes and cultured at room temperature (25° C.) for 7 days.

Then, 10 mL of distilled water and 0.5 ml of the three artificial humic acids (KOH, KOH+FeCl₃ and KOH+Fe₂O₃) were respectively added to the culture dishes and cultured for 7 days.

### Comparative Example 3

The addition of the artificial humic acids was omitted, and the other conditions were the same as in Example 2. Seeds were germinated, which was recorded as the blank group (CK).

During the seed germination process of Example 2 and Comparative Example 3, the germination rates were recorded. The test results are shown in Table 1:

TABLE 1
Effects of adding artificial humic acids on germination rate of rice

| | Germination rate (%) | | | | | | | |
|---|---|---|---|---|---|---|---|---|
| Treatment | Day 0 | Day 1 | Day 2 | Day 3 | Day 4 | Day 5 | Day 6 | Day 7 |
| CK | 0 ± 0a | 0 ± 0b | 62.5 ± 3b | 67.5 ± 2d | 67.5 ± 4c | 70.0 ± 3d | 80.0 ± 6d | 85.0 ± 2c |
| KOH | 0 ± 0a | 0 ± 0b | 55.0 ± 5c | 77.5 ± 6c | 82.5 ± 3b | 86.7 ± 4c | 90.0 ± 2c | 95.0 ± 1b |
| KOH + FeCl₃ | 0 ± 0a | 0 ± 0b | 62.5 ± 0b | 80.0 ± 2ab | 80.0 ± 5bc | 90.0 ± 1bc | 95.0 ± 1b | 100.0 ± 4a |
| KOH+ Fe₂O₃ | 0 ± 0a | 6.0 ± 1a | 70.0 ± 1a | 85.0 ± 2a | 92.5 ± 4a | 100.0 ± 4a | 100.0 ± 3a | 100.0 ± 1a |

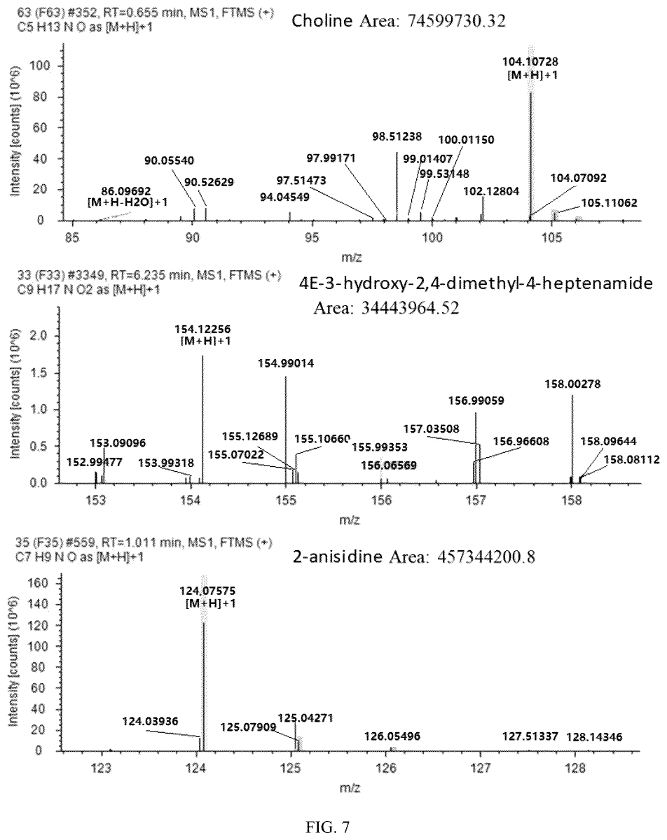

As can be seen from FIG. 7 and Table 1, the artificial humic acid synthesized with catalysis of the nanoscale ferric oxide (KOH+Fe₂O₃ treatment group) can accelerate germination of rice seeds and improve the germination rate of rice seeds by 15%.

### Example 3 Rice Growth

A method for promoting rice growth by using artificial humic acid included the following steps:

Rice seeds were germinated in a vermiculite culture medium. The seeds with uniform germination conditions were selected and placed in a 100 mL PVC test tube. 60 g of soil and 2 mL of artificial humic acids (which were the KOH treatment group, the KOH+FeCl₃ treatment group and the KOH+Fe₂O₃ treatment group) were added. The seeds were cultured for 30 days. During the culture, the seeds were exposed to light for 12 h every day, and the day and night temperatures were 25° C. and 20° C. respectively.

### Comparative Example 4

The addition of the artificial humic acids was omitted, and the other conditions were the same as in Example 3. The rice was grown, which was recorded as the blank group (CK).

After the completion of the culture, the plant height, dry weight, fresh weight, nutrient contents, photosynthetic rate, soluble sugar content and soluble protein content were determined.

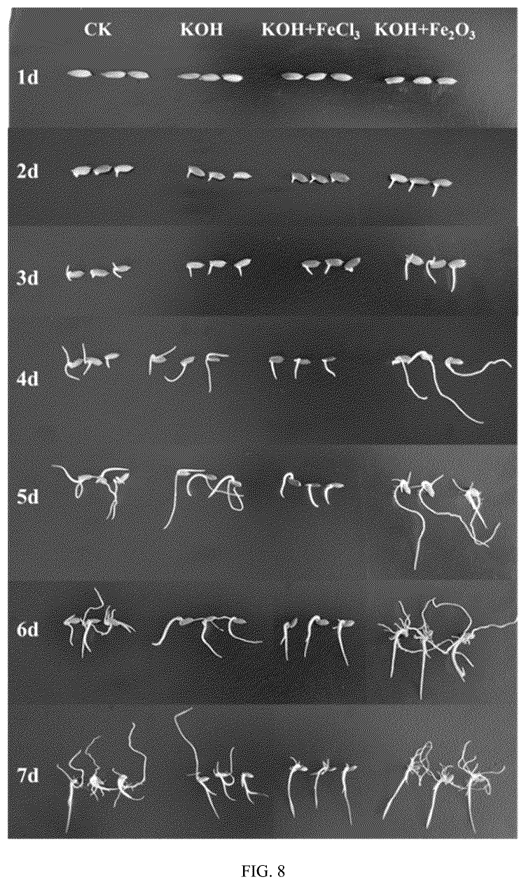

As can be seen from FIG. 8 and Table 2 to Table 5, the artificial humic acid synthesized with catalysis of nanoscale ferric oxide improves the plant height of rice by 89.50%, the root length by 50.80% (Table 2), the root activity by 166.76% and the net photosynthetic rate by 72.08% (Table 3), and significantly improves the abilities of rice to absorb and transport nutrients (Table 4 and Table 5).

TABLE 2
Plant height, root length, biomass content and water content of rice

| | Length (cm) | | Fresh weight (g) | | Water content (%) | |
|---|---|---|---|---|---|---|
| Treatment | Aerial | Roots | Aerial parts | Roots | Aerial parts | Roots |
| CK | 8.67 ± 3.1c | 13.1 ± 2.0b | 0.084 ± 0.009a | 0.057 ± 0.009a | 77.149 ± 3.4a | 68.710 ± 3.4b |
| KOH | 12.6 ± 2.6b | 20.0 ± 1.2a | 0.097 ± 0.003a | 0.070 ± 0.006a | 82.167 ± 1.8a | 76.589 ± 1.4ab |
| KOH+ | 13.2 ± 1.8b | 20.3 ± 3.5a | 0.093 ± 0.003a | 0.077 ± 0.017a | 77.492 ± 1.1a | 76.753 ± 3.4ab |
| KOH+ | 16.43 ± 2.4a | 19.8 ± 3.3a | 0.099 ± 0.006a | 0.065 ± 0.003a | 80.926 ± 0.9a | 78.687 ± 0.5a |

Note:
Different lowercase letters indicate significant differences between the 4 treatments (P < 0.05).

TABLE 3
Photosynthetic and root activity indexes of rice

| | Net | | | |
|---|---|---|---|---|
| | photosynthetic | Transpiration | | |
| | rate | rate | Root volume | Root activity |
| Treatment | (umol CO₂ m⁻²s⁻¹) | (mmol H₂O m⁻²s⁻¹) | (cm³) | (ug*g⁻¹*h⁻¹) |
| CK | 6.57 ± 0.23b | 3.05 ± 0.20b | 0.06 ± 0.02b | 45.45 ± 1.13c |
| KOH | 7.37 ± 0.48b | 2.97 ± 0.19b | 0.09 ± 0.01a | 58.79 ± 2.73b |
| KOH + FeCl₃ | 10.40 ± 0.80a | 3.79 ± 0.40ab | 0.09 ± 0.01a | 67.27 ± 4.22b |
| KOH + Fe₂O₃ | 11.30 ± 0.45a | 4.34 ± 0.24a | 0.10 ± 0.01a | 121.25 ± 4.70a |

Note:
Different lowercase letters indicate significant differences between the 4 treatments (P < 0.05).

TABLE 4
Nutrient contents of rice

| | Soluble sugar (%) | | Soluble protein (mg*g⁻¹) | |
|---|---|---|---|---|
| Treatment | Aerial parts | Roots | Aerial parts | Roots |
| CK | 14.16 ± 0.90d | 10.66 ± 1.13c | 451.07 ± 5.40a | 289.76 ± 2.18b |
| KOH | 25.27 ± 1.73c | 18.18 ± 1.11b | 405.23 ± 10.52a | 291.83 ± 8.35b |
| KOH + FeCl₃ | 55.75 ± 0.65a | 35.81 ± 0.80a | 445.63 ± 27.45a | 335.49 ± 35.78ab |
| KOH + Fe₂O₃ | 45.87 ± 1.92b | 36.07 ± 1.53a | 461.32 ± 16.38a | 363.21 ± 18.10a |

TABLE 5
Nutrient utilization of rice

| | N (plant/mg) | | P (plant/mg) | | K (plant/mg) | |
|---|---|---|---|---|---|---|
| Treatment | Aerial | Roots | Aerial | Roots | Aerial parts | Roots |
| CK | 0.56 ± 0.01a | 1.57 ± 0.11a | 5.35 ± 0.51b | 16.44 ± 3.53a | 0.26 ± 0.01bc | 2.55 ± 0069a |
| KOH | 0.35 ± 0.02b | 0.90 ± 0.01b | 4.98 ± 0.23b | 13.02 ± 0.97a | 0.25 ± 0.01c | 1.20 ± 0.07b |
| KOH + | 0.37 ± 0.02b | 0.87 ± 0.00b | 7.21 ± 0.53a | 14.66 ± 2.79a | 0.29 ± 0.01b | 1.14 ± 0.21b |
| FeCl₃ | | | | | | |
| KOH + | 0.31 ± 0.02b | 0.91 ± 0.19b | 7.47 ± 0.49a | 11.67 ± 1.28a | 0.34 ± 0.01a | 1.41 ± 0.07ab |
| Fe₂O₃ | | | | | | |

Note:
Different lowercase letters indicate significant differences between the 4 treatments (P < 0.05).

### Comparative Example 5

The KOH in Example 1 and Comparative Examples 1 and 2 were replaced with HCl, and the other conditions were the same as in Example 1 to obtain artificial humic acids.

The artificial humic acids in Example 1 and Comparative Examples 1, 2 and 5 were compared. The results are shown in Table 6.

As can be seen from Table 6, the humic acids synthesized under alkaline conditions has higher TOC contents, and the addition of the catalyst can improve the TOC content.

TABLE 6
Test results of TOC

| Treatment | TOC content (g L⁻¹) |
|---|---|
| KOH | 7.93 ± 1.10b |
| KOH + FeCl₃ | 6.04 ± 0.59c |
| KOH + Fe₂O₃ | 9.10 ± 0.76a |
| HCl | 5.02 ± 0.85d |
| HCl + FeCl₃ | 0.39 ± 0.10f |
| HCl + Fe₂O₃ | 4.47 ± 0.98de |

Note:
Different lowercase letters indicate significant differences between the treatments (P < 0.05).

Based on the above, in the synthesis of artificial humic acid with catalysis of the nanoscale ferric oxide according to the present disclosure, the degradation rate of biomass in the hydrothermal process is improved by more than 14%, and the synthesized artificial humic acid contains phytohormone substances such as coumaric acid and isocitric acid. This not only improves the preparation efficiency of the artificial humic acid and the contents of beneficial components in the product, but also significantly promotes germination and growth of rice. This technique of promoting rice growth by using the artificial humic acid synthesized with catalysis of the nanoscale ferric oxide can effectively avoid the accumulation of waste biomass and environmental hazards, and realize the backflow and neutralization of the soil carbon pool, which is of great significance for the development of green agriculture and the mitigation of global climate crisis.

What is claimed is:

1. A method for synthesizing artificial humic acid from waste biomass with catalysis of transition metal, comprising the following steps:
adding a mixed solution of an alkali, a transition metal catalyst and water into a biomass raw material, and carrying out catalytic reaction at 160-250° C. for 6-48 hours; after the completion of the reaction, carrying out solid-liquid separation on a reaction product, and filtering an obtained liquid to obtain the artificial humic acid;
wherein the transition metal catalyst is nanoscale ferric oxide; and
a ratio of the biomass raw material to the transition metal catalyst is 2.5 to 3.5:1.

2. A method for synthesizing artificial humic acid from waste biomass with catalysis of transition metal, comprising the following steps:
adding a mixed solution of an alkali, a transition metal catalyst and water into a biomass raw material, and carrying out catalytic reaction at 160-250° C. for 6-48 hours; and after the completion of the reaction, carrying out solid-liquid separation on a reaction product, and filtering an obtained liquid to obtain the artificial humic acid.

3. The method according to claim 2, wherein the biomass comprises bulk farmland waste biomass and greening waste biomass; wherein the bulk farmland waste biomass comprises straws, rice husks and peanut shells; and the greening waste biomass comprises branches and leaves.

4. The method according to claim 2, wherein the transition metal catalyst is nanoscale ferric oxide.

5. The method according to claim 2, wherein a ratio of the biomass raw material to the transition metal catalyst is 2.5:1 to 3.5:1.

6. The method according to claim 2, wherein the prepared artificial humic acid contains coumaric acid and isocitric acid.

7. A method for promoting plant growth by using artificial humic acid, comprising: adding the artificial humic acid in a process of plant seed germination or plant growth and carrying out a culture; and
wherein a method for preparing the artificial humic acid comprises the following steps:
adding a mixed solution of an alkali, a transition metal catalyst and water into a biomass raw material, and carrying out catalytic reaction at 160-250° C. for 6-48 hours; and after the completion of the reaction, carrying out solid-liquid separation on a reaction product, and filtering an obtained liquid to obtain the artificial humic acid.

8. The method according to claim 7, wherein the adding the artificial humic acid in the process plant seed germination comprises: culturing plant seeds in a solution with the artificial humic acid at room temperature until the plant seeds germinate.

9. The method according to claim 8, comprising sterilizing the plant seeds before germination; and wherein the sterilization uses a 5% (v:v) H₂O₂ solution.

10. The method according to claim 7, wherein the adding the artificial humic acid in the process of plant growth comprises: culturing plant seeds with uniform germination conditions in an environment containing soil and the artificial humic acid; and wherein a ratio of the artificial humic acid to the soil is 1/30 (mL/g).
